(19) United States
(12) Patent Application Publication
(10) Pub. No.: US 2011/0152876 A1
(43) Pub. Date: Jun. 23, 2011

(54) TOOL FOR AFFIXING A MARK DRIVING A ROTATION OF THE PUNCH USED TO PIERCE THE SKIN OF THE ANIMAL

(75) Inventor: Bernard Vandeputte, Boulogne Billancourt (FR)

(73) Assignee: Allflex Europe

(21) Appl. No.: 12/937,917

(22) PCT Filed: Apr. 3, 2009
(86) PCT No.: PCT/EP09/54033

§ 371 Date: Feb. 22, 2011

(30) Foreign Application Priority Data
Apr. 14, 2008 (FR) 0852496

(51) Int. Cl. A61B 17/00

(52) U.S. Cl. 606/116

(57) ABSTRACT

The invention relates to a tool for placing a mark for animal marking, said mark combining by nesting: a male portion (2), including a rod defining a fitting axis and enabling the guidance of an awl (11) for piercing the skin of the animal, and a female portion (5), including a recess for engaging said rod. According to the invention, the tool includes a means for setting said awl (11) in rotation about said fitting axis.

### FIELD OF THE INVENTION

The field of the invention is that of identifying and marking animals.

More precisely, the invention relates to the perforation of the tissues of an animal, for example on its ear, during the installation of a device for marking, still called a mark, and where applicable the sampling of tissues of the animal. Such samplings make it possible in particular to retain cells carrying biological or biochemical characteristics of the animal, for example in order to later identify the animal or detect diseases, by analysing its DNA.

### PRIOR ART

The identification of cattle has been made mandatory in many countries in order to provide for the monitoring of the herd and to guarantee the origin of the animals intended in particular for consumption. This marking relates to bovines as well as ovines, porcines, goats, winged creatures, or any other animal species.

Traditionally, a mark includes an identification number inscribed on a label making it possible to follow the animal all throughout its existence. For example, in accordance with European Directive 1760/2000, all bovines must be identified by an ear mark affixed to each ear, the two marks bearing the same unique identification code which makes it possible to identify each animal individually, as well as the farm where it is born. These marks must therefore be tamper proof in order to provide for the traceability of the animal, i.e. it must not be possible to remove a mark affixed on a first animal in order to re-affix it on a second animal.

Plastic marks currently used most often. They include a loop bearing a label whereon is mentioned all of the legal information via impression or engraving, via laser marking, or any other means known to those skilled in the art. They are in particular designed to be affixed irreversibly, using a tool or “tagger” (for example pliers) for tamper-proof attaching, associating in a snap locking arrangement a male component passing through the ear of the animal and a female component.

For example, as shown in a simplified manner in FIGS. 1A and 1B, such a mark 1 associates via nesting a male component 2 comprising a pin 3 and a female component 5 comprising a receiving button 6.

More precisely, the pin 3 of the male component 2 extends from a support 10 intended to bear on a face of the ear of the animal, perpendicularly (or substantially perpendicularly) to this support. This pin 3 is terminated by a tip 4.

As shown in FIG. 1A, the receiving button 6 of the female component 5 has an orifice 7 and a cavity 8 for the introduction of the tip 4 of the pin 3. The base of the female component can bear against the other face of the ear.

During the placing of the mark 1, the tip 4 of the pin 3 is guided into the receiving button 6 through the skin of the animal, for example the ear, in such a way as to associate the male 2 and female 5 components in a snap locking arrangement.

Once the mark 1 has been installed, the tip 4 is housed in the cavity 8, and is maintained therein by a retaining shoulder 9 realising the transition between the cavity 8 and the orifice 7. It is therefore no longer possible to remove the mark, or to modify it, without breaking it once it has been installed.

As shown in FIG. 1B, the receiving button 6 can also be open. This is in this case referred to as a mark, or loop, with an open head.

Such marks are carriers of small marked plates or labels identifying the animal. In particular, the button can comprise a small plate offering a marking surface for the identification of the animal.

It is also known to take tissue samples from the animal during the placing of a mark 1, as described in patent application FR 2 906 703 in the name of the same Applicant.

To do this, as shown in FIG. 2A, the pin 3 is hollow, and allows for the passage of a punch 11. This punch 11 can be removed after the placing of the mark 1. Such a punch 11 has a cutting edge 12, of generally circular shape, making it possible to perforate the skin 13 of the animal during the placing of the mark. For example, the cutting edge 12 can be of continuous contact, as shown in FIG. 2B, or toothed or saw-toothed, as shown in FIG. 2C.

As such, during the association of the male 2 and female 5 components, the punch 11 perforates the skin 13 of the animal along a circumferential line of contact, and guides the tip 4 of the pin 3 into the receiving button 6. In addition, the sample of tissues cut out by the cutting edge 12 remains imprisoned in the punch 11, and can be recovered after the punch 11 is removed.

Due to the thickness of the tissues to pierce during the placing of the ear mark, it is necessary that the edge 12 of the punch be very sharp so that the sample of tissues remains imprisoned in the punch 11. Indeed, the ear of the animal is comprised of several layers including, in a simplified manner, a first skin, cartilage, and a second skin, called “skin” in what follows. In addition, the punch 11 cannot bear on a hard surface during the placing of the ear mark, since it passes through the skin of the animal and enters into the receiving button 6.

Unfortunately, this technique does not make it possible to fully ensure the success of each sampling. Indeed, from time to time, the sample of tissues cut out by the punch 11 is not entirely detached from the ear of the animal. For example, the punch 11 makes it possible to cut out the first skin and the cartilage, but does not always make it possible to correctly cut out the second skin. The sample can then remain, in the female component 5 or the male component 2, connected to the ear by a “filament”.

Consequently, when the punch 11 is removed the latter is empty and the sample is lost. As the mark has already been installed, it is not possible to start the operation again, and it is necessary to carry out a separate operation if it is desired to take a tissue sample from the animal.

Therefore, there is a need for a new technique making it possible to overcome this problem while making it possible to recover in an almost certain manner a sample of tissues in the punch, in relation to known techniques.

### DESCRIPTION OF THE INVENTION

The invention propose a new solution which does not have all of these disadvantages of prior art, in the form of a tool for affixing (placing) a mark for animal marking, said mark associating in a snap locking arrangement:

a male component comprising a pin defining a fitting axis, and making it possible to guide a punch intended to pierce the skin of the animal, and

a female component comprising a cavity intended to interact with said pin.

According to the invention, the tool comprises means for setting in rotation said punch around said fitting axis.

This rotation of the punch around the fitting axis makes it possible to improve the quality of the cut out of the skin of the animal, during the placing of the mark. In this way, all of the layers of the skin (first skin, cartilage, second skin, etc.) are correctly cut out and the sample of tissues cut out by the punch is fully detached from the animal.

As such, the setting in rotation of the punch makes it possible to recover in an almost certain manner a sample of tissues from the animal during the installation of the mark, while according to prior art, the samples could remained blocked in the mark when the punch is removed.

It can be noted that the solution proposed can apply regardless of the type of mark used for the sampling of tissues of the animal.

As such, according to a first type of mark, the pin of the male component is hollow and allows for the passage of the punch intended to pierce the skin of the animal. The punch then takes the form of a cylindrical shaped sampling needle, having a sharp edge, for example with continuous or toothed contact, and the means for setting in rotation make is possible to drive the punch in rotation, independently of the male component.

According to a second type of mark, the punch is attached to the male component. For example, the punch is nested on the end of the pin of the male component. The punch then takes the form of a cylindrical-shaped sampling receptacle, having a sharp edge, for example with continuous or toothed contact, and the means for setting in rotation make it possible to drive the male component in rotation (or in any case at least the pin), as well as the punch attached to the pin.

According to a particular aspect of the invention, the tool comprises at least one mobile branch that can be actuated manually, acting on the means for setting the punch in rotation or on means of driving the means for setting the punch in rotation.

In this way, it is not necessary for the person installing the mark to carry out additional actions compared to the techniques of prior art. Indeed, it is the normal movement of one or several mobile branches which guides, directly or indirectly, the rotation of the punch.

For example, the tool for affixing the mark is of the pliers type, comprising two articulated branches. These branches act on two jaws, intended to carry respectively the male component and the female component of the mark. These two jaws can have two extreme positions, an open position, making it possible to place the male and female components on either side of the location where it is desired to affix the mark (ear, wing or skin of the animal for example), and a closed position, when the jaws are actuated in order to associate the male and female portions. The passage from one position to the other, actuated manually by at least one branch, drives a rotation of the punch, by acting directly on the means for setting the punch in rotation or on means of driving the means for setting the punch in rotation.

In particular, the means of driving belong to the group including:

a connecting rod attached by a first of its ends to a branch of the tool and by a second of its ends to the means for setting in rotation;

a rack attached on the one hand to a branch of the tool and on the other hand to the means for setting in rotation.

As such, the drawing closer of the two branches during the installation of the mark can drive a connecting rod, which in turn drives the means for setting the punch in rotation. In this way, the operations carried out by the person who is affixing the mark are similar and of likewise complexity as those carried out according to the techniques of prior art.

According to a particular aspect of the invention, the means for setting the punch in rotation include a nesting housing able to interact with a complementary form provided for this purpose on the punch.

For example, it is considered that the punch comprises two portions: a first portion making it possible to collect a sample of tissues from the animal, such as a sampling (or biopsy) needle, and a second portion making it possible to interact with the means for setting in rotation. For example, this second portion comprises protrusions, intended to interact with at least one drive groove present in the nesting housing.

According to a first embodiment, the means for setting the punch in rotation include at least one toothed wheel.

Such a toothed wheel can include a nesting housing taking for example the form of an extruded cylindrical portion, comprising a grooved bore. Once the punch is nested in the nesting housing, the rotation of the toothed wheel drives the rotation of the punch.

Advantageously, such a wheel is driven by a drive wheel, having a greater diameter, itself driven by means of driving, such as a connecting rod or a rack, attached to a branch of the tool for example.

As such, the drawing closer of the branches during the installation of the mark can drive the connecting rod, which drives the drive wheel, which in turn drives the driven wheel, and the rotation of the punch. In this way, the operations of perforating the skin, and of rotating the punch, can be implemented simultaneously.

According to a second embodiment, the means for setting the punch in rotation include a guiding axis having a helical groove, and a fixed finger making it possible to transmit a rotation movement to said guiding axis, in such a way as to drive the rotation of the punch during the placing of the mark.

For example, the punch is attached to the guiding axis, located at least partially in a housing of the tool. Due to the fact that the fixed finger is inserted in the helical groove of the guiding axis, the guiding axis begins to rotate when it exits its housing. It therefore drives the punch in rotation.

In this way, the operations of perforating the skin, and of rotating the punch, can be implemented simultaneously.

According to a third embodiment, the means for setting the punch in rotation include at least one shaft intended to receive said punch in its extension, and attached to the tool by the intermediary of a ring.

Such a ring makes it possible to maintain the shaft in its axis of rotation, while leaving it free to rotate.

In particular, the shaft comprises a thumbwheel attached to one of its ends. As such, a rotation of the thumbwheel generates a rotation of the shaft, which generates in turn a rotation of the punch.

According to this third embodiment, the rotation of the punch can be implemented after the skin of the animal has been perforated.

According to a fourth embodiment, the tool for affixing the label comprises means of motorisation acting on the means for the setting the punch in rotation. For example, these means of motorisation include a reduction gear motor driving a driver, whereon is attached the punch.

In particular, the tool comprises means of switching controlling the means of motorisation.

According to a particular characteristic, these means of switching are actuated by at least one branch of the tool for affixing. As such, when at least one of the branches is actuated manually in order to place the mark, the means of switching are acted on, which authorise the operation of the means of motorisation, and drive the rotation of the punch. In this way, the operations of perforating the skin, and of rotating the punch, can be implemented simultaneously.

### LIST OF FIGURES

Other characteristics and advantages of the invention shall appear more clearly when reading the following description of a particular embodiment, provided as a simple non-limiting example, and of the annexed drawings, wherein:

FIGS. 1A and 1B, already presented in relation with prior art, show the conventional structure of an ear mark;

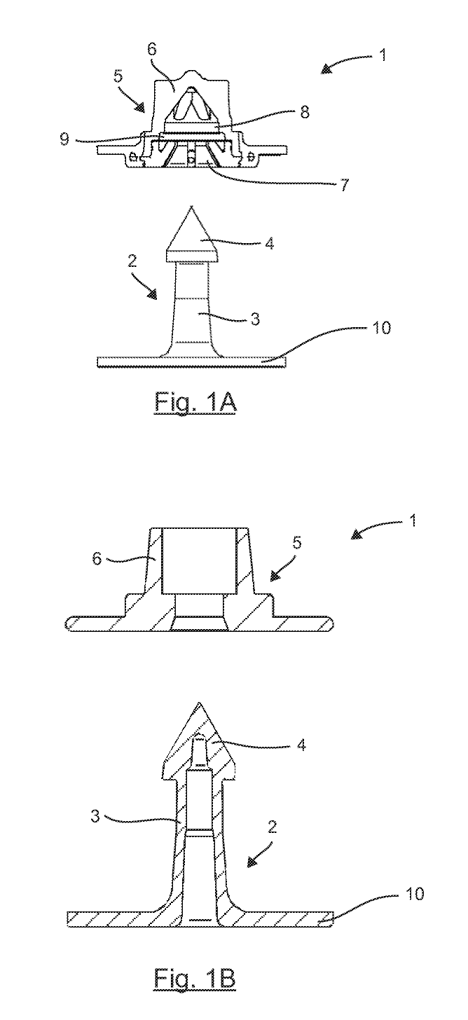

FIGS. 2A to 2C, also described previously, show an ear mark making it possible to carry out a sampling of tissues according to prior art;

FIGS. 3A to 3D show the general principle of the invention;

FIGS. 4A and 4B show a first embodiment of the invention;

FIGS. 5A and 5B propose a second embodiment;

FIGS. 6A to 6C show a third embodiment; and

FIGS. 7A and 7B show a fourth embodiment.

### DESCRIPTION OF AN EMBODIMENT OF THE INVENTION

### General Principle

The general principle of the invention is based on a modification of the movement of the punch used to perforate the skin of an animal during the installation of a mark, with the purpose in particular of taking tissue samples from the animal. This new movement of the punch makes it possible to effectively cut out the tissues from the ear of the animal or from the location on the body of the animal whereon the mark is affixed.

More precisely, the invention proposes to apply a rotation movement to the punch during the placing of the mark, in such a way as to improve the quality of the cut out of all of the layers of the ear of the animal (first skin, cartilage, second skin, etc., called “skin” in what follows). In this way in an almost certain manner a sample of tissues of the animal is recovered in the punch. This rotation can be implemented independently of the male portion of the mark, in particular in the case where the punch takes the form of a needle sliding in the hollow pin of the male portion, or result from a rotation of the male portion of the mark, in particular in the case where the punch takes the form of a receptacle attached to the end of the pin of the male portion.

Further more precisely, this rotation movement is generated by the tool for affixing the ear mark, or “tagger”. In other words, the tagger comprises means for setting the punch in rotation around the fitting axis defined by the hollow pin of the male portion.

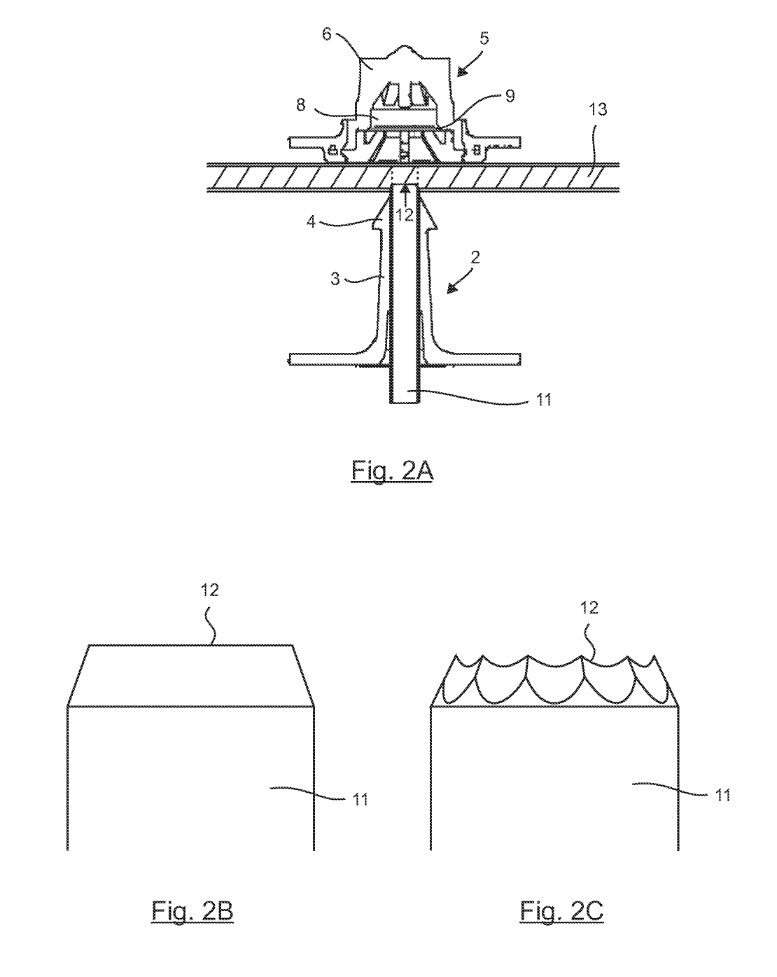

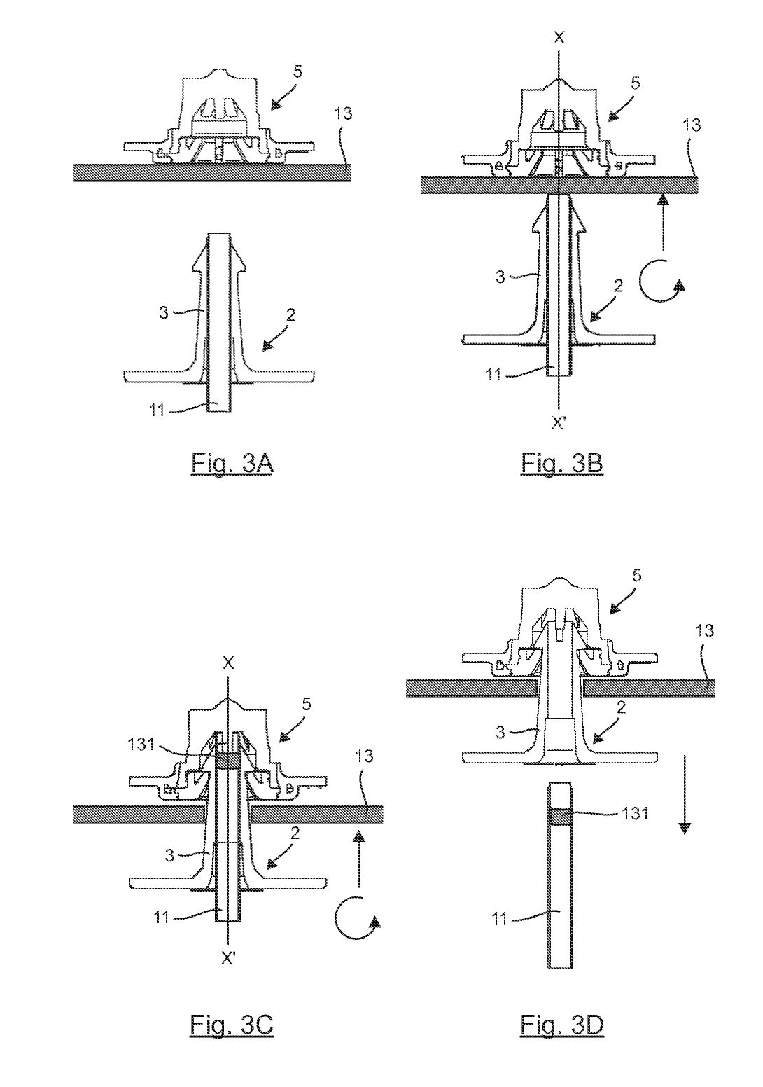

FIGS. 3A to 3D show this general principle, for the placing of an ear mark similar to that described in relation with prior art, in FIG. 2A. It can be noted that several alternatives of the structure of this mark are known. Of course, the invention is not limited to the installation of a mark having the structure described previously, but also applies to these alternatives.

As such, as shown in FIG. 3A, the male portion 2 and the female portion 5 are arranged on each side of the ear 13 of the animal to be marked or of the location on the body of the animal whereon the mark is to be affixed. For example, the female portion 5 is maintained on a first jaw of the tagger for attaching the mark, and the male portion 2 is maintained on a second jaw of the tagger.

When the jaws tighten, as shown in FIG. 3B, the punch 11 comes into contact with the skin of the animal, and pierces the ear 13. More precisely, the punch 11 undergoes a double translation movement when it sinks into the ear of the animal, and of rotation around the fitting axis XX′ when it cuts out the tissues of the ear, as shown by the arrows. Of course, these two movements are not necessarily carried out in a simultaneous manner, but can be implemented one after the other, with a translation movement first in order to pierce the ear of the animal, then a rotation movement in order to properly cut out the tissues. Note that a low rotation (of a magnitude of one revolution for example) is sufficient to cut all of the tissues.

As shown in FIG. 3C, this double movement of translation and of rotation can continue until the nesting (or the snap-fitting) of the male 2 and female 5 portions. In this way, the complete cut out of the sample 131 of tissues from the ear is ensured, regardless of the thickness of the ear of the animal. In addition, when the punch 11 comes to abut in the bottom of the cavity of the female component 5, the sample 131 is slightly pushed back into the punch 11, in order to prevent the sample 131 from remaining blocked in the cavity of the female component.

It is then possible to remove the punch 11 from the pin 3 of the mark once it has been installed, as shown in FIG. 3D, then to recover the sample of tissues 131 located in the punch 11.

The sample of tissues 131 can then be stored in a test tube (or micro test tube), and sent to a laboratory for storage and possibly analysis.

### DESCRIPTION OF DIFFERENT EMBODIMENTS

Hereinafter we shall focus on describing a tagger comprising means for setting the punch in rotation according to the invention, making it possible to install a mark on the ear of an animal, for example of the bovine, ovine, porcine, goat, or winged-creature type, or any other animal species. Of course, other locations on the body of the animal can also be considered for affixing a mark with the proposed tagger, for example on the wing for a winged-creature.

### First Embodiment

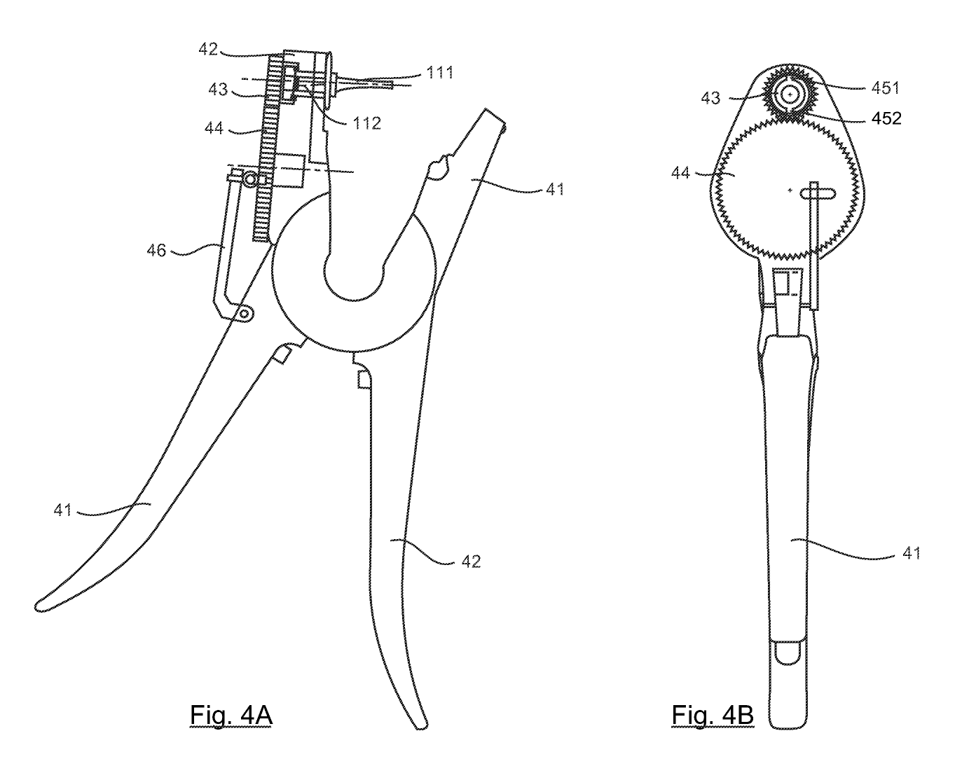

According to a first embodiment shown in FIGS. 4A and 4B a tool of the pliers type is considered, comprising two branches 41, 42, each connected to a jaw, also referenced as 41, 42. This is for example pliers of the “Total Tagger” type (registered trademark).

The female component of the mark is for example attached to the jaw 41, and the male component to the jaw 42. More precisely, the male component is nested on the punch 11, which can be directly attached to the jaw 42, or threaded on a center punch which is itself attached to the jaw 42. In particular, the punch 11 has an adapted shape in order to slide at least partially in the hollow pin of the male component of the mark.

For example, the punch 11 comprises a sampling needle 111, having a cylindrical shape, and means of attaching 112 to the jaw 42. These means of attaching 112 can include protrusions.

According to this first embodiment, the means for setting the punch in rotation include a gearing formed of two toothed wheels 43 and 44, possibly superimposed. Of course, a different number of gearings or wheels can also be considered.

For example, the toothed wheel 43, referred to as the driven wheel, comprises a nesting housing able to interact with a complementary form provided for this purpose on the punch. This nesting housing takes for example the form of an extruded cylindrical component, comprising a grooved bore. The grooves or notches 451, 452 of the nesting housing interact with the protrusions of the means of attaching 112 of the punch, in such a way as to maintain the punch 11, and to guide it in rotation. In this way, a rotation of the driven wheel 43 generates a rotation of the punch 11, around the axis of the punch.

The wheel 44, referred to as the drive wheel, has a diameter greater than the driven wheel 43, and makes it possible to drive the driven wheel 43 in rotation. The drive wheel 44 is driven by means of driving 46. For example, these means of driving 46 include a connecting rod attached by a first of its ends to the branch 41 of the pliers and by a second of its ends to the drive wheel 44. According to an alternative, these means of driving include a rack attached on the one hand to the branch 41 of the pliers and on the other hand to the drive wheel 44. Of course, these means of driving are not necessarily apparent and could be incorporated into the interior of the pliers, for example in a branch.

In this way, an action on the branches 41, 42 of the pliers in order to close the jaws acts on the means of driving 46 the drive wheel 44, which drives in rotation the driven wheel 43, which in turn drives the punch in rotation around its axis.

According to an alternative not shown, the punch 11 has an adapted shape in order to nest at the end of the pin of the male component of the mark.

According to this alternative, the means for setting the punch in rotation make it possible to drive the male component in rotation. For example, the male component is attached to the jaw 42 by threading the pin of the male component on a center punch which itself is attached to the jaw 42. The setting in rotation of the drive 44 and driven 43 wheels then drives the rotation of the center punch, which drives the rotation of the male component and therefore of the punch nested on the pin of the male component.

### Second Embodiment

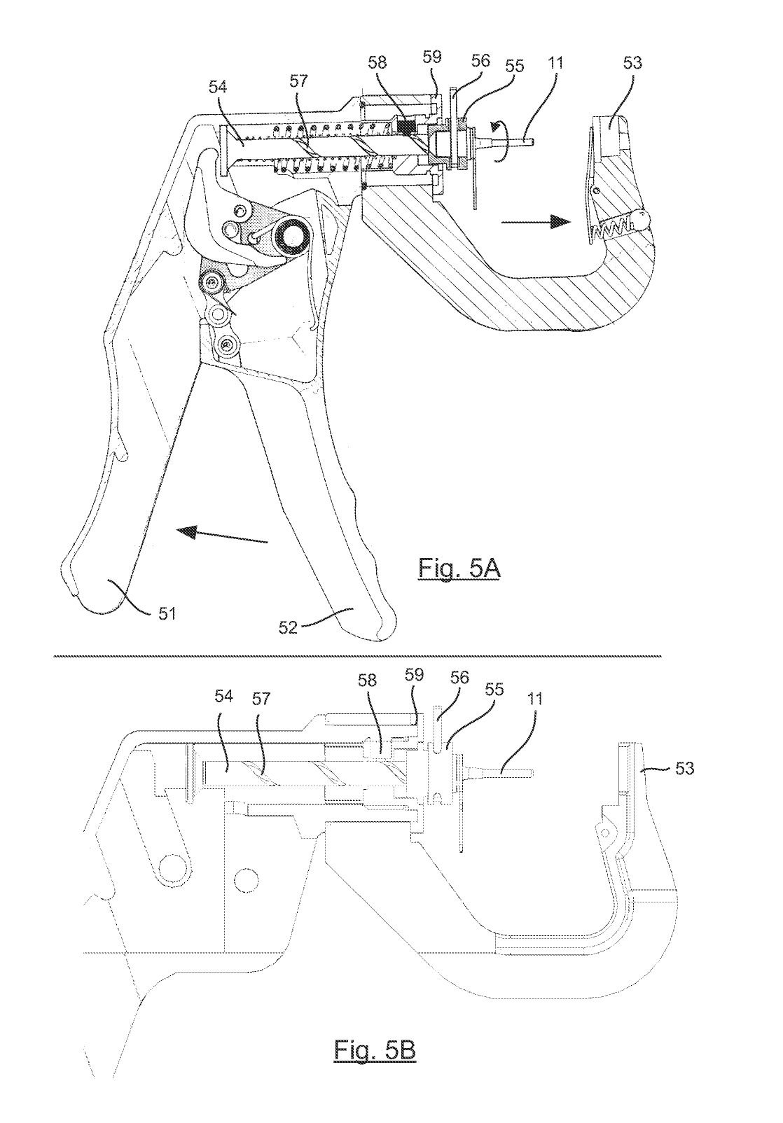

Hereinafter is described a second embodiment, shown in FIGS. 5A and 5B, wherein the tool for affixing is of the pliers type comprising two branches 51, 52. The second branch 52 is a mobile branch that can be actuated manually, acting on means of driving the means for setting the punch in rotation.

The female component of the mark is for example attached to a jaw 53 used as an anvil. The male component is nested on the punch 11, which has an adapted shape in order to slide at least partially in the hollow pin of the male component of the mark.

The punch 11 is maintained at the end of a guiding axis 54 in a support 55, by moveable means of blocking 56. These means of blocking 56 take for example the form of a clamping plate. It is as such easy to change the punch 11, by removing the means of blocking 56.

The support 55 is for example maintained on the tool for affixing by the intermediary of a guiding ring 59. Such a ring makes it possible to maintain the support 55, while leaving it free to rotate.

According to this embodiment, the guiding axis 54 takes the form of a worm screw, having a helical groove 57 wherein a finger 58 comes to be housed, attached to the carcass of the pliers.

The guiding axis 54 is initially located at least partially in a housing of the pliers.

When the second branch 52 is actuated, in such a way as to draw closer the two branches 51 and 52 according to the direction of the solid-line arrow, the guiding axis 54 is impacted, and the finger 58 inserted in the groove 57 transmits a rotation movement to the guiding axis 54 (the finger being fixed).

The punch 11 then undergoes a double movement of translation towards the jaw 53 bearing the female component, and of rotation according to the direction of the solid-line arrows. The setting in rotation of the punch 11 then makes it possible to effectively cut the different tissues from the ear of the animal, and in particular the first skin, the cartilage, and the second skin.

According to an alternative not shown, the punch 11 has an adapted shape in order to nest at the end of the pin of the male component of the mark.

According to this alternative, the means for setting the punch in rotation make it possible to drive the male component in rotation. For example, the male component is attached to the guiding axis 54 by threading the pin of the male component on a center punch which itself is attached to the guiding axis 54. The setting in rotation of the guiding axis 54 by the finger 58 during the percussion of the guiding axis 54 then drives the rotation of the center punch, which drives the rotation of the male component and therefore of the punch nested on the pin of the male component.

### Third Embodiment

According to a third embodiment, the movements of displacement of the punch towards the male component (translation or rotation around the axis of the pliers) and rotation of the punch around its axis are decorrelated.

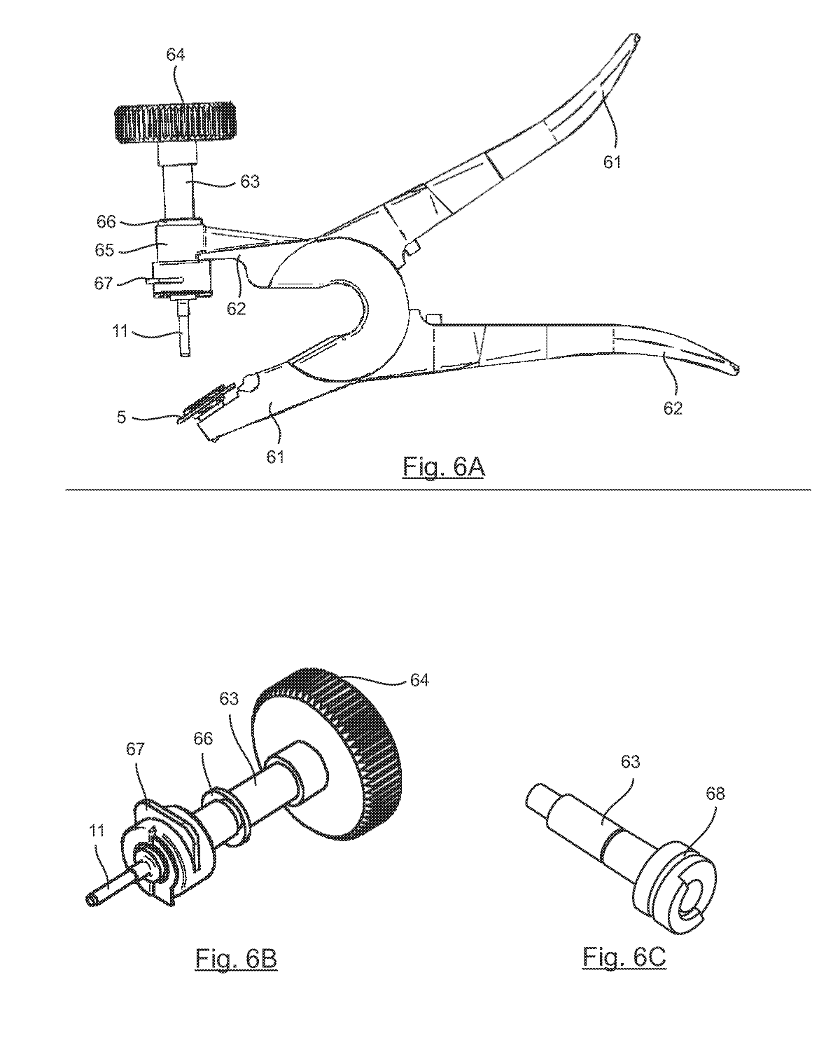

More precisely, as shown in FIGS. 6A to 6C, the tool for affixing is of the pliers type comprising two branches 61, 62, each connected to a jaw, also referenced as 61, 62.

The female component 5 of the mark is for example attached to the lower jaw 61. The male component is nested on the punch 11, attached to the upper jaw 62. Again, the punch 11 has an adapted shape in order to slide at least partially in the hollow pin of the male component of the loop.

According to this third embodiment, the means for setting the punch 11 in rotation around the fitting axis defined by the hollow pin of the male component include a shaft 63 intended to receive the punch 11 in its extension. The shaft further comprises a thumbwheel 64 attached to one of its ends. The thumbwheel 64 is dimensioned according to the desired degree of rotation.

More precisely, the shaft 63 is maintained on the pliers by means of a ring, for example a bronze or retaining ring 65, or any other means making it possible to maintain the shaft 63 on the pliers while still facilitating its rotation.

For example, the shaft 63 is provided with a locking ring 66 making it possible to maintain the shaft 63 in the bronze or retaining ring 65.

The shaft 63 has a housing adapted to interact with the shape of the punch 11. It comprises for example a recess corresponding to the shape of a small plate of the punch 11, making it possible to drive the punch 11 during the rotation of the shaft 63.

The punch 11 can be maintained at the end of the shaft 63 by movable means of blocking 67. These means of blocking 67 take for example the form of a plate, which can be inserted into the groove 68. It is as such easy to change the punch 11, by removing the means of blocking 67.

According to this embodiment of the invention, the ear of the animal is first perforated by closing the jaws 61 and 62 when the branches 61, 62 of the pliers are actuated.

Then, the thumbwheel 64 is turned manually. The rotation of the thumbwheel 64 generates the rotation of the shaft 63 in the bronze or retaining ring 65, which drives the rotation of the punch 11. The setting in rotation of the punch 11 then makes it possible to cut the last filaments connecting the sample of tissues to the ear of the animal. A low rotation, of a magnitude of one revolution for example, is sufficient to obtain a cut out of better quality than according to prior art.

According to an alternative, the rotation of the thumbwheel 64 can also generate a screwing/unscrewing movement. In this way, the two movements of perforation and of rotation can be carried out simultaneously.

This embodiment can also be adapted to drive the rotation of the male component of the mark, when the punch has an adapted shape in order to nest at the end of the pin of the male component of the mark.

### Fourth Embodiment

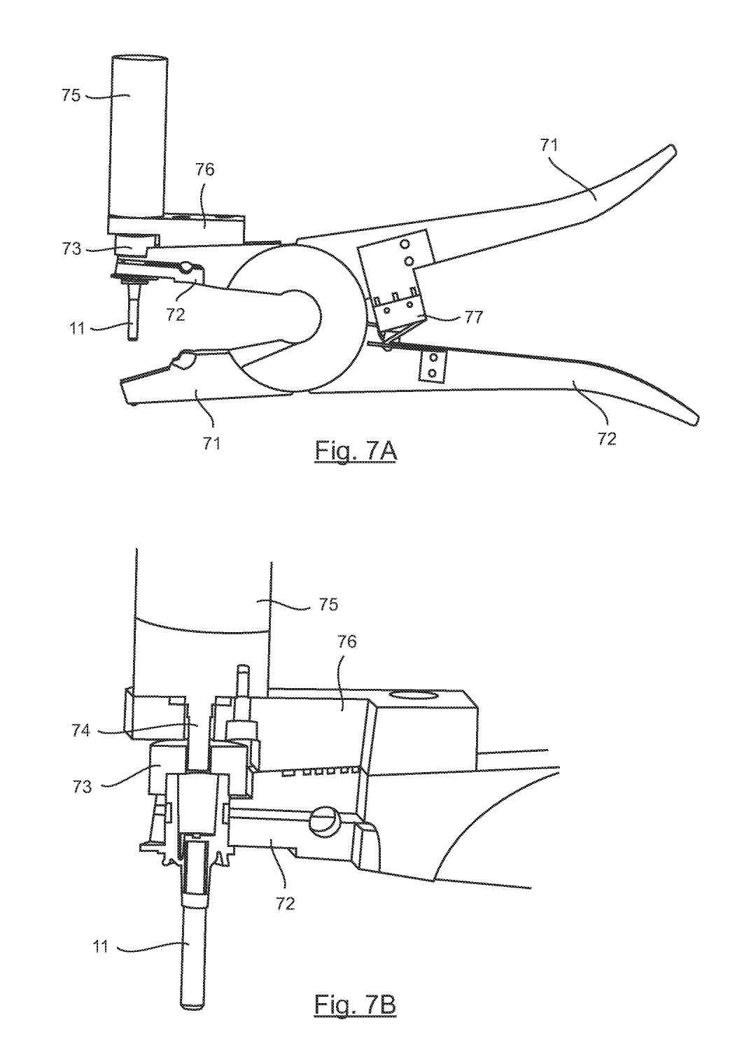

FIGS. 7A and 7B show a fourth embodiment according to which the tool for affixing comprises means of motorisation acting on the means for setting the punch in rotation. These means of motorisation can be placed in the axis of the punch, or perpendicularly to the axis of the punch, in such a way as to reduce the encumbrance.

For example, as shown in FIG. 7A, the tool for affixing a mark is of the pliers type comprising two branches 71, 72, each connected to a jaw, also referenced as 71, 72.

The female component of the mark is for example attached to the lower jaw 71. The male component is nested on the punch 11, attached to the upper jaw 72 by the intermediary of a driver 73 and a reduction gear motor 75. Again, the punch 11 has an adapted shape in order to slide at least partially in the hollow pin of the male component of the mark.

According to this embodiment, the punch 11 interacts with the driver 73.

For example, the driver 73 comprises a nesting housing able to interact with a complementary form provided for this purpose on the punch, such as grooves.

This driver 73 is attached with a shaft 74 controlled by the reduction gear motor 75. In this way, the activation of the reduction gear motor 75 drives the rotation of the shaft 74, which drives the rotation of the driver 73, which drives the rotation of the punch 11. The reduction gear motor 75 is for example attached to the jaw 72 by means of attaching taking the form of metal plates.

According to an alternative, the tool for affixing an ear mark according to the invention comprises means of switching 77 controlling the means of motorisation.

These means of switching are for example controlled by the branches 71 and 72. As such, a drawing closer of the branches 71 and 72 in order to tighten the jaws creates a contact on means of switching 77, generating an electric current which triggers the activation of the reduction gear motor 75.

Such a reduction gear motor can also be powered by a battery or an exterior current. For example, it can be activated as long as the jaws 71 and 72 of the pliers are tightened, or only in such a way as to turn the punch 11 a half-turn or a full turn.

This embodiment can also be adapted in order to drive the rotation of the male component of the mark, when the punch has an adapted shape in order to nest at the end of the pin of the male component of the mark.

Of course, the different embodiments are purely for the purposes of illustration, and are not limiting. In particular, it is possible to combine the various embodiments considered.

1. Tool for affixing a mark for animal. marking, said mark associating in a snap locking arrangement:
a male component (2) comprising a pin defining a fitting axis, and making it possible to guide a punch (11) intended to pierce the skin (13) of the animal., and
a female component (5) comprising a cavity intended to interact with said pin,
characterised in that said tool. comprises means for setting said punch (11) in rotation around said fitting axis.

2. Tool according to claim 1, characterised in that it comprises at least one mobile branch, able to be actuated manually, acting on said means for setting the punch (11) in rotation or on means of driving said means for setting the punch (11) in rotation.

3. Tool according to claim 2, characterised in that said branch or branches also act on two jaws, intended to carry said male component (2) and said female component (5) respectively.

4. Tool according to claim 2, characterised in that said means of driving belong to the group comprising:
a connecting rod attached by a first of its ends to a branch of said tool. and by a second of its ends to said means for setting in rotation;
a rack attached on the one hand to a branch of said tool and on the other hand to said means for setting in rotation.

5. Tool according to claim 1, characterised in that said means for setting said punch (11) in rotation include a nesting housing able to interact with a complementary form provided for this purpose on said punch (11).

6. Tool according to claim 5, characterised in that said nesting housing comprises at least one drive groove of said punch (11).

7. Tool according to claim 1, characterised in that said means for setting said punch (11) in rotation include at least one toothed wheel. (43, 44).

8. Tool. according to claim 1, characterised in that said means for setting said punch (11) in rotation include a guiding axis (54) having a helical groove (57) and a fixed finger (58) making it possible to transmit a rotation movement to said guiding axis.

9. Tool according to claim 1, characterised in that said means for setting said punch in rotation include at least one shaft (63) intended to receive said punch in its extension, and attached to said tool by the intermediary of a ring.

10. Tool according to claim 9, characterised in that said shaft comprises a thumbwheel (64) attached to one of its ends.

11. Tool according to claim 1, characterised in that it comprises means of motorisation acting on said means for setting said punch in rotation.

12. Tool according to claim 11, characterised in that it comprises means of switching controlling said means of motorisation (77).

13. Tool according to claim 11, characterised in that it comprises at least one branch acting on said means of switching.

14. Tool according to claim 1, characterised in that said punch (11) is attached to said male component (2), and in that said means for setting said punch (11) in rotation drive said male component (2) in rotation around said fitting axis.
